(19) United States
(12) Patent Application Publication
(10) Pub. No.: US 2015/0148600 A1
(43) Pub. Date: May 28, 2015

(54) MEDICAL INSTRUMENT

(71) Applicant: CANON KABUSHIKI KAISHA, Tokyo (JP)

(72) Inventors: Takaaki Ashinuma, Tokyo (JP); Takahisa Kato, Brookline, MA (US)

(73) Assignee: Canon Kabushiki Kaisha, Tokyo (JP)

(21) Appl. No.: 14/403,987

(22) PCT Filed: May 17, 2013
(86) PCT No.: PCT/JP2013/003154

§ 371 Date: Nov. 25, 2014

(30) Foreign Application Priority Data
May 31, 2012 (JP) 2012-124505
Jul. 31, 2012 (JP) 2012-169756

(51) Int. Cl. A61B 1/00; A61B 1/04; A61B 1/005

(52) U.S. Cl. CPC A61B 1/00006; A61B 1/0008; A61B 1/0016; A61B 1/0057; A61B 1/04; A61B 1/05
USPC 600/109; 604/95.04

(57) ABSTRACT

The present invention provides a medical instrument which is capable of reducing damage, such as cutting of a wire, to the medical instrument even if excessively large load is applied to an inserting portion. A medical instrument including: a deformable portion; a wire configured to deform the deformable portion; a driving unit configured to drive the wire; a drive control unit configured to control the driving unit; and a load detecting unit configured to detect load applied to the deformable portion, wherein when the load detected by the load detecting unit exceeds a threshold value, the drive control unit controls the driving unit to retain posture of the deformable portion.

### TECHNICAL FIELD

The present invention relates to a medical instrument which includes a controllable bending portion, such as an endoscope and a catheter.

### BACKGROUND ART

A medical device, such as an endoscope and an electrophysiological catheter, which passes through a structure of a living body, such as a body cavity, and accesses a target location includes an inserting portion which is inserted in a patient's body. Some medical devices include a bendable bending portion in the inserting portion which may follow the structure of the living body.

Operability may be increased by guiding the device to various locations of the living body using a bending function.

In such a related art device, an operation wire is attached to a bendable structure and, when the operation wire is drawn by a driving unit, a bending operation is performed.

Regarding performing the bending operation inside the body cavity, the following have been proposed: a related-art rigid endoscope which may detect contact with the body cavity; a retreat of a bendable endoscope; and a process in a case in which external load is applied to a treatment tool in which a sheath like the endoscope is used. PTL 1 describes an endoscope which includes a contact detecting unit which generates a signal upon contact. PTL 2 describes an endoscope which includes a tactile sensor and is capable of detecting load applied to a portion of the endoscope which is inserted in a body cavity and capable of detecting contact of a tip of the endoscope with an internal organ. PTL 3 describes a medical treatment instrument which keeps a state of a treatment unit even when external force is added to the treatment unit.

When a bendable medical device is used, since, for example, a thin material is used in a small-sized endoscope, there is a possibility that damage is caused to a medical instrument due to overload if excessively large load is applied to the inserting portion.

The present invention provides a medical instrument which is capable of reducing damage, such as cutting of a wire, to the medical instrument even if excessively large load is applied to an inserting portion.

### CITATION LIST

### Patent Literature

PTL 1: Japanese Patent Laid-Open No. 2010-175962
PTL 2: Japanese Patent Laid-Open No. 2008-017903
PTL 3: Japanese Patent Laid-Open No. 2007-44330

### SUMMARY OF INVENTION

The present invention provides a medical instrument including: a deformable portion; a wire configured to deform the deformable portion; a driving unit configured to drive the wire; a drive control unit configured to control the driving unit; and a load detecting unit configured to detect load applied to the deformable portion, wherein when the load detected by the load detecting unit exceeds a threshold value, the drive control unit controls the driving unit to retain posture of the deformable portion.

Further features of the present invention will become apparent from the following description of exemplary embodiments with reference to the attached drawings.

### Advantageous Effects of Invention

According to the present invention, a medical instrument in which damage, such as cutting of a wire, caused to the medical instrument may be reduced by retaining, by a posture retaining unit, posture of the inserting portion even when excessively large load is applied to a deformable portion may be provided.

### BRIEF DESCRIPTION OF DRAWINGS

FIG. 1A is a side view illustrating a configuration of a medical device according to one embodiment of the present invention.

FIG. 1B is a side view illustrating an operation of a medical device according to one embodiment of the present invention.

FIG. 2 is a block diagram illustrating a configuration of a medical device according to one embodiment of the present invention.

FIG. 3 is a conceptual diagram illustrating a state in which the medical device according to one embodiment of the present invention is in contact with a peripheral portion.

FIG. 4 is a flowchart related to an embodiment of the present invention.

FIG. 5 is a cross-sectional view of a tip portion load detecting unit according to one embodiment of the present invention.

FIG. 6 is a flowchart related to an embodiment of the present invention.

FIG. 7 is a conceptual diagram of one embodiment of the present invention.

FIG. 8 is a block diagram of a current detecting unit according to one embodiment of the present invention.

FIG. 9 is a conceptual diagram of one embodiment of the present invention.

FIG. 10 is a block diagram of one embodiment of the present invention.

### DESCRIPTION OF EMBODIMENTS

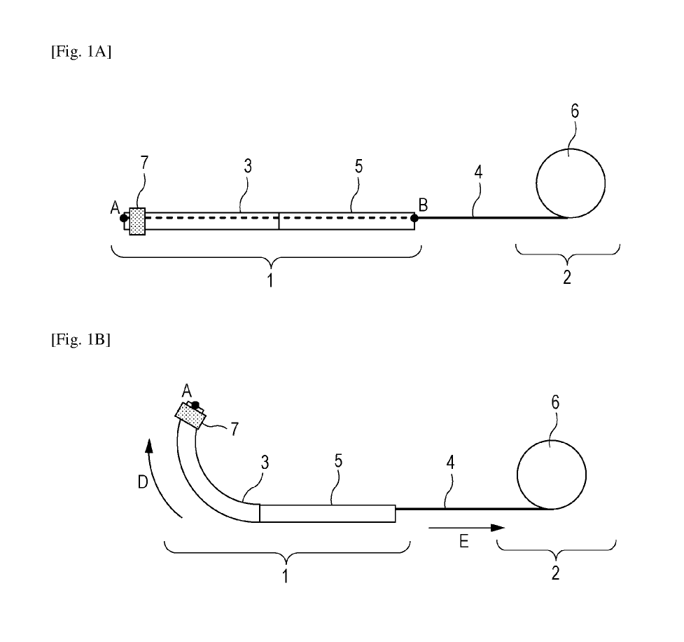

As illustrated in FIGS. 1A and 1B, a medical instrument according to one embodiment of the present invention includes a bending portion 3 which is a deformable portion, a non-bending portion 5, and a wire 4 (hereafter, referred to as a control wire). The wire receives driving force from a driving pulley 6 which is a driving unit. A tactile sensor 7 which is a load detecting unit is provided at a tip of the bending portion 3. An inserting portion 1 includes the bending portion 3 and the non-bending portion 5.

In response to an instruction from an unillustrated drive control unit, driving force is transmitted to the wire from the driving unit and the wire is driven.

Load applied to the deformable portion may be detected by the load detecting unit. When the load exceeds a threshold value, the drive control unit controls the driving unit to retain posture of the deformable portion. That is, when the load exceeds a threshold value, the inserting portion is controlled to retain posture at the time when the load exceeds the threshold value.

The load detecting unit may be, for example, a measuring unit which measures pressure, a measuring unit which measures size of a driving current, or a measuring unit which measures tension. A single or a plurality of load detecting units may be provided.

A means to retain posture may be, for example, to continuously transmit the same driving force as the driving force applied when the load exceeds the threshold value to the driving unit.

Hereinafter, a medical device according to one embodiment of the present invention will be described with reference to a preferred embodiment.

The medical instrument according to the present embodiment includes a configuration illustrated in FIGS. 1A and 1B. Relationships among components of the medical device of the present invention are illustrated in the side view of FIG. 1A. The medical instrument of the present embodiment includes an inserting portion 1 which may be inserted in a narrow space, such as a body cavity. The inserting portion 1 includes a tip portion illustrated as a point A.

The inserting portion has an elongated cylindrical shape in the direction from the point A to a point B. Hereafter, the side of the point A will be referred to as a tip side and the side of the point B, which is the side opposite to the point A, will be referred to as a base end side.

The inserting portion 1 may be used as an endoscope in which an image pickup unit, an illuminating unit and the like are mounted at the tip portion thereof or may be used as an electrophysiological catheter in which an electrode is disposed at the tip portion thereof.

If the inserting portion 1 is used as an endoscope which includes an image pickup optical system at the tip thereof, the tip includes a portion for taking light information of an object. The image pickup optical system which takes the light information may be, for example, an objective lens, optical fiber and a light transmission window for observation.

Light guided by the image pickup optical system of the endoscope is picked by an image pickup element disposed inside or outside of a medical instrument body. It is also possible to provide an image pickup element, such as a semiconductor image sensor, at the tip and perform image pickup at an observation unit.

The illuminating unit of the endoscope may use light which is emitted from a light source disposed inside or outside of the medical instrument body and is guided by, for example, optical fiber. Alternatively, the illuminating unit may include, for example, an LED at the tip thereof for illumination.

The tactile sensor 7 which detects that the tip portion has brought into contact with a peripheral portion is provided at the tip portion. The tactile sensor has four divided areas along a circumferential direction of the tip portion and the direction and a value of the applied load may be calculated on the basis of values detected in the four areas.

One end of the control wire 4 is fixed to the tip portion and the other end of the control wire 4 is fixed to a driving unit 2. The control wire 4 is a wire material which is bendable and by which tension may be transmitted.

The control wire passes through the inserting portion 1 as illustrated by the broken lines. An unillustrated guide hole is formed in the inserting portion 1 at the portion of the control wire 4 illustrated by the broken line so that the control wire 4 may be moved in the longitudinal direction thereof.

The position in which the control wire 4 is inserted is disposed in the inserting portion outside the center of a section of the inserting portion 1. The control wire may be disposed along a surface of the inserting portion.

The driving unit 2 is connected to an unillustrated power source. In this manner, tractive force from the power source is transmitted to the control wire 4 via the driving unit 2.

The inserting portion 1 includes the bending portion 3 and the non-bending portion 5. The bending portion 3 is a portion which is bent by the wire 4.

The non-bending portion 5 is a portion which is not bent even when the wire 4 is drawn. Although the bending portion 3 is disposed at the tip side and the non-bending portion 5 is disposed at a base end side in the illustration, arrangement thereof are not limited to the same. Alternatively, a plurality of bending portions may be provided via or not via the non-bending portion.

The non-bending portion 5 may be a rigid portion which is hardly deformed or may be a bendable flexible portion (rigidity in the bending direction is greater than that of the bending portion 3).

The driving mechanism 20 includes the wire 4 and a driving pulley 6 as a driving unit. The driving pulley 6 is connected to a driving source. When the driving pulley 6 is rotated, the wire 4 may be taken up and drawn.

The driving force provided to the wire is not limited to tractive force. In a case in which the wire is an electronic device of which longitudinal dimension is changed by a current, the driving force may be a current.

The wire 4 is made of a member which transmits tractive force. The wire 4 may be a wire material which is bendable and by which tension is transmitted. The driving unit 2 may have other configuration which transmits tractive force from the driving source. For example, the driving unit 2 may be a column-shaped member which may be pressed and drawn.

Next, a bending operation of the medical instrument according to the present embodiment will be described with reference to FIG. 1B. The driving pulley 6 takes the wire 4 up in the direction of an arrow E and the wire 4 is drawn.

The wire 4 is fixed to the tip portion A of the inserting portion. In addition, the wire 4 is inserted in the deformable portion outside the center of a section of the deformable portion.

Therefore, tension produced when the control wire 4 is drawn becomes torque which causes the bending portion 3 to be bent in the direction of an arrow D. The bending portion 3 is bent as illustrated due to the bending torque.

The size of the bending torque may be controlled by controlling an amount of rolling up of the driving pulley 6. In this manner, the bending operation of the bending portion 3 may be controlled.

Desirably, the medical instrument according to the present embodiment further includes an inserting portion shape detecting unit. Since it is possible to detect the shape of the inserting portion, usability is increased.

The entire configuration of one embodiment of the medical instrument of the present invention will be described with reference to a block diagram of FIG. 2.

A load detecting unit 11 which is, for example, a tactile sensor, is provided at the tip of the inserting portion 1. The load detecting unit 11 sends load information 14 at the tip of the inserting portion to a controller 13 which controls the entire system.

During normal operation, the controller 13 calculates a driving control signal 18 on the basis of position information (not illustrated) regarding a position at which the tip portion should exist and an inserting portion shape signal 15 sent from an inserting portion shape detecting unit 12, and then issues an instruction to a drive control unit 17.

In response to the instruction, the drive control unit 17 sends a driving signal 19 to a driving mechanism 20 and drives the pulley 6 of a driving mechanism 20 illustrated in FIG. 1 so that the tip of the inserting portion is moved to a target position.

The controller 13 monitors an output of the load detecting unit 11 at the tip of the inserting portion, determines whether dynamic load at the tip of the inserting portion is equal to or smaller than a tolerance, and controls an operation of the inserting portion in accordance with the determination result.

### First Embodiment

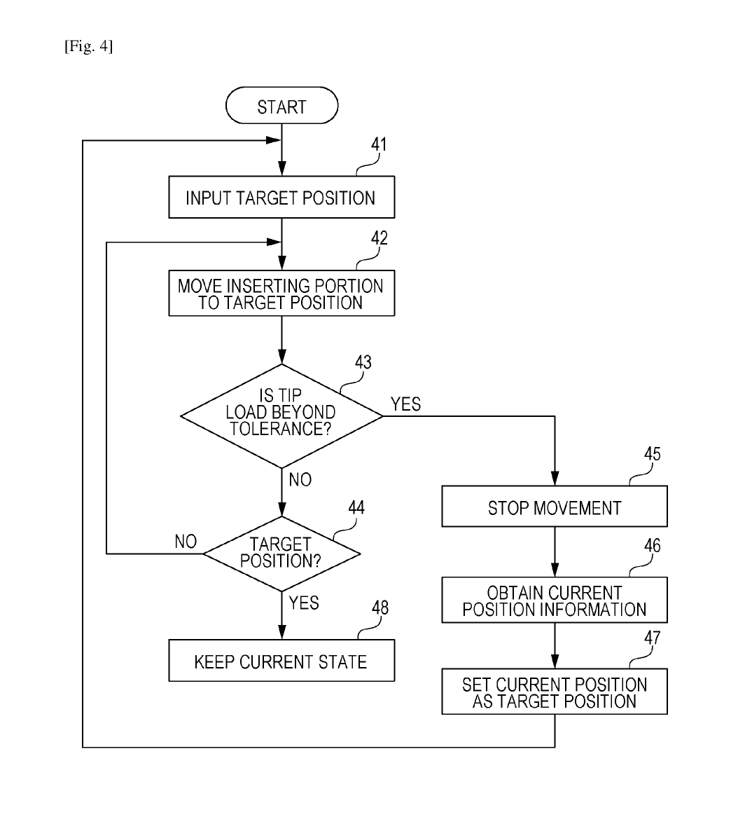

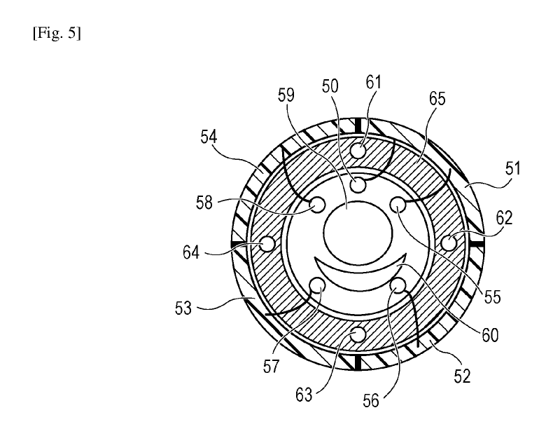

Next, with reference to flowcharts of FIGS. 4 and 6, an operation in a case in which an output of the inserting portion load detecting unit 11 exceeds a tolerance while the tip of the inserting portion is being moved will be described.

A target position is input from an input device (not illustrated) connected to the controller 13 (step 41) and the inserting portion 1 starts movement toward the target position (step 42).

The controller 13 monitors the output of the load detecting unit 11 at the tip of the inserting portion and determines whether dynamic load at the tip of the inserting portion is equal to or smaller than the tolerance (step 43).

Here, in a case in which the tip of the inserting portion is not in contact with a peripheral portion or, in a case in which contact pressure is equal to or smaller than a tolerance even if the tip of the inserting portion is in slight contact with the peripheral portion (step 43: NO), it is determined whether the current position has been the target position on the basis of the information about the inserting portion shape detecting unit 12 (step 44).

If the current position has not been the target position (step 44: NO), the movement toward the target position is continued. The inserting portion shape detecting unit 12 is incorporated in a driving mechanism 20 which drives the tip of the inserting portion and calculates the position of the tip of the inserting portion and the shape of a middle portion on the basis of a driving amount of the wire.

The detecting unit of the driving amount of the wire may be, for example, a means to provide a physical scale on the wire and to optically detect a moved amount of the wire.

Alternatively, the detecting unit of the driving amount of the wire may be a means to add an encoder to a pulley which drives the wire or to a motor for driving and to calculate the driving amount of the wire.

Another method for detecting the shape of the inserting portion may include, for example, a magnetic field system in which the shape of the inserting portion is detected directly and the position is known.

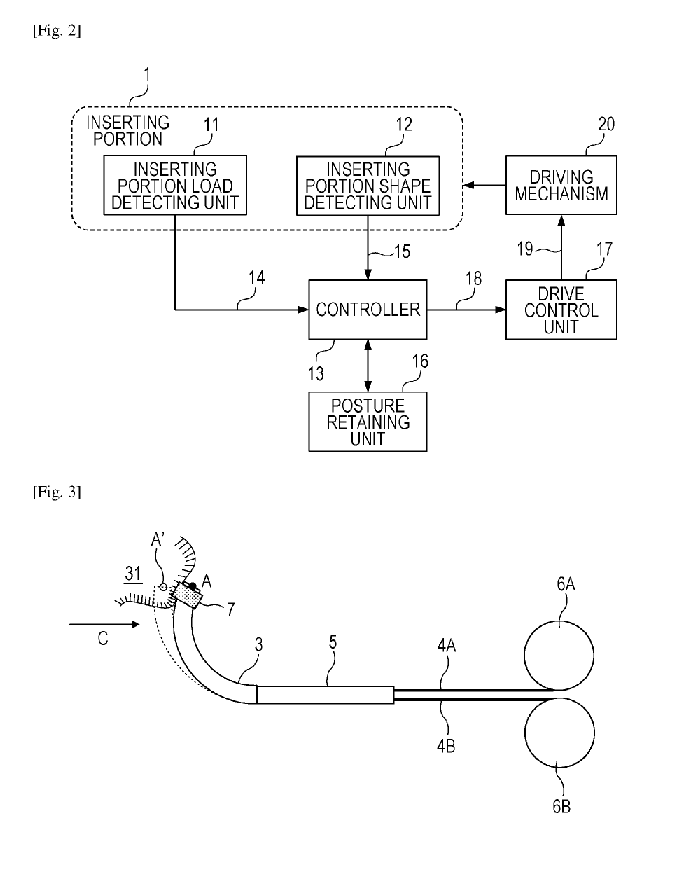

FIG. 3 illustrates a state in which guidance of the inserting portion has not been performed precisely due to, for example, difference between a preoperative image at the tip of the inserting portion and an actual position and the inserting portion has been in contact with the peripheral portion.

The tip of the inserting portion should be at the position of the point A′ in a normal situation, but is pressed in the direction of an arrow C due to the contact with peripheral tissue 31 and has been at the position of the point A.

The drive control unit 17 controls the inserting portion so that the position of the tip of the inserting portion becomes the position of A′ and, therefore, larger load than usual is applied to the tip of the inserting portion.

Therefore, a wire 4B on the extension side of the driving mechanism 20 is drawn by the external force and there is a possibility of cutting of the wire 4B. At this time, the tactile sensor 7 provided at the tip of the inserting portion receives force from the direction of the arrow C.

In a case in which the load detecting unit is a measuring unit which measures pressure, it is desirable that a plurality of the load detecting units are disposed at the tip of the deformable portion. The plurality of the load detecting units are arranged spaced from one another. Such a configuration is desirable because information about the direction in which the load is applied may be obtained by the plurality of load detecting units.

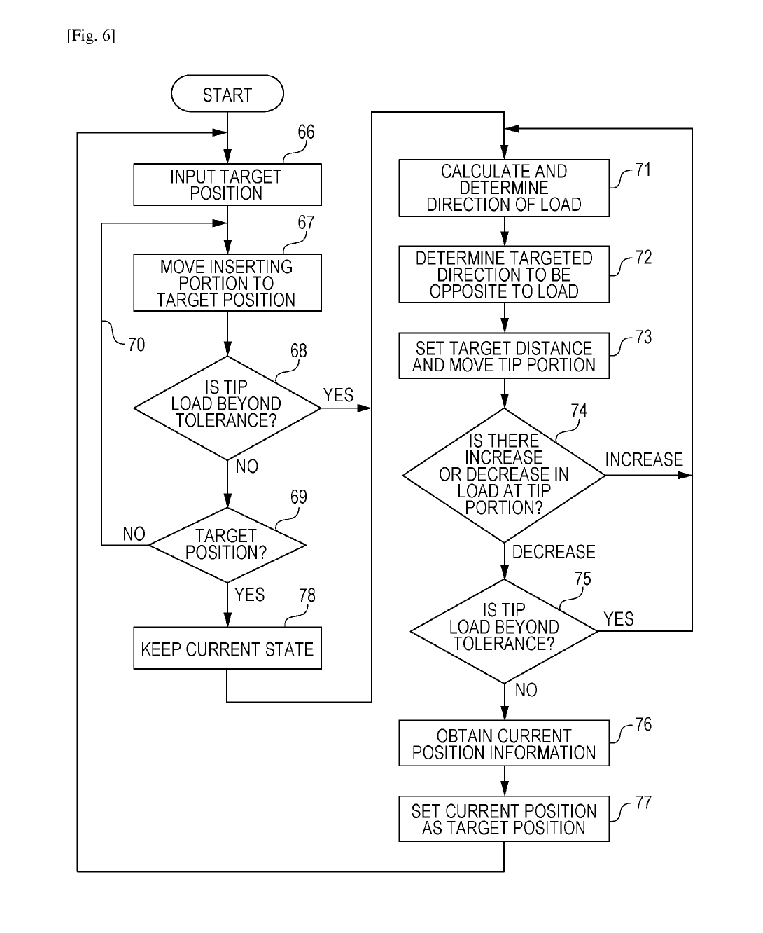

An exemplary configuration in which a plurality of load detecting units are provided is illustrated in a cross-sectional view of FIG. 5 in which the tactile sensor 7 is provided at the tip of the inserting portion. The tactile sensor 7 is made of a conductive resin material which has four areas 51, 52, 53 and 54 along the circumferential direction at the tip of the inserting portion.

Resistance values of the four areas are changed in accordance with load applied thereto. Detected values, i.e., the amount of change of resistance, of each area are measured. The direction and the value of the applied load may be computed by calculating in an internal computing unit (not illustrated) of the controller 13.

An output of the tactile sensor 7 is transmitted to the controller 13 by conductive members 55, 56, 57 and 58 which pass through the medical instrument. The reference sign 50 denotes a conductive member for common power supply for tactile sensors 51, 52, 53 and 54, 59 denotes an optical fiber bundle for image observing, 60 denotes optical fiber for illumination, 61, 62, 63 and 64 denote wire guides and 65 denotes a sheath body.

Although the tactile sensor 7 here is a sensor which uses resistance change with respect to pressure, the system of the tactile sensor 7 is not particularly limited. For example, a sensor using the MEMS technique and the change in electric capacity may also be used.

The tactile sensor 7 corresponds to the load detecting unit 11 in the block diagram of FIG. 2. If the load applied to the inserting portion exceeds a tolerance when, for example, the inserting portion is brought into contact with a peripheral portion, an output of the load detecting unit 11 is calculated in an overload determination unit (not illustrated) which is incorporated in the controller 13 and it is determined that the size of the load has exceeded a predetermined tolerance (step 43: YES).

Almost at the same time, when it is determined, by the overload determination unit which is incorporated in the controller 13, that the size of the load has exceeded the tolerance, the controller 13 instructs the drive control unit 17 to stop the movement (step 45).

Here, a parameter necessary to retain the current posture is calculated in a posture retaining unit 16 on the basis of information 15 from the inserting portion shape detecting unit 12 and current position information is obtained (step 46).

The obtained current position information is set to be the target position (step 47). An instruction is issued to the drive control unit on the basis of the parameter necessary to retain the posture. The driving unit is controlled so that the current posture of the insert portion is kept and the insert portion is stopped at the current position.

### Second Embodiment

Next, an operation in a case in which the peripheral portion has moved due to a certain change of state while the inserting portion is in a stationary state and is controlled to fix the position thereof will be described.

In a state in which the posture of the deformable portion is retained by the drive control unit, when the load detected by the load detecting unit exceeds the threshold value, it is desirable that the drive control unit causes the deformable portion to deform so as to reduce the load. At this time, the load detecting unit is desirably a measuring unit which measures pressure.

During an operation of the change in the shape of the inserting portion, when the inserting portion is brought into contact with the peripheral portion, retention of the posture may avoid problems in a case in which the peripheral portion is not moved; but in a case in which the position of the peripheral portion is varied, a collision avoidance operation is necessary since retention of the current posture is insufficient to avoid problems.

An operation when overload is applied while the inserting portion is in a stationary state will be described with reference to a flowchart of FIG. 6. The flowchart of FIG. 6 illustrates a state in which the inserting portion is in a stationary state (i.e., a state under control to keep a predetermined posture).

A target position in the stationary state is set (step 66) and the stationary state is kept by controlling the inserting portion to move toward the target position (step 67).

Next, it is determined whether the load at the tip is equal to or greater than a tolerance (step 68). If the load is below the tolerance (step 68: NO), the state is kept and the position of the tip of the inserting portion is controlled to be the target position (step 69, loop 70).

If the load at the tip of the inserting portion is equal to or greater than the tolerance (step 68: YES), the detection result of the sensor which is divided into four constituting the tactile sensor 7 is calculated by a calculating unit (not illustrated) which is incorporated in the controller 13 as described above and the strength and the direction of the force applied to the tip of the inserting portion are calculated (step 71).

Although the procedure in this process is described to calculate the strength and the direction of the force for every loop in the present embodiment, it is also possible to always perform the calculating operation to obtain the strength and the direction of the force all the time.

In that case, data about the strength and the direction of the force applied to the tip portion is obtained in step 71.

When the strength and the direction of the force applied to the tip of the inserting portion are determined, a direction in which the tip of the inserting portion is moved is set to be the direction opposite to the direction of the force applied to the tip of the inserting portion. That is, it is determined that the tip of the inserting portion is moved in the direction in which the external force applied at the tip of the inserting portion becomes small, i.e., in the direction of a vector which includes no component of the direction opposite to the component of the direction which the vector of the external force applied to the tip of the inserting portion includes and, preferably, in the same direction as the direction which the vector of the external force applied to the tip of the inserting portion includes (step 72). Then a predetermined target distance is set and the tip of the inserting portion is controlled to move (step 73). In this manner, the driving unit is controlled to deform the deformable portion so that the load detected by the load detecting unit becomes small. The load detecting unit is disposed at the tip of the inserting portion in the present embodiment. However, also in a case in which the load detecting unit is disposed between the tip and the base end of the deformable portion as in a sixth embodiment which will be described later, the driving unit may be controlled in the manner described above to deform the deformable portion in the direction in which the load detected by the load detecting unit becomes small.

The distance here is preferably set in accordance with details of treatment and sites to which the medical instrument according to the present embodiment is applied, and other environmental conditions.

After the inserting portion is moved, the load applied to the tip portion is determined (step 74). If the load applied to the tip portion is increased and the load exceeds the tolerance, the routine returns to step 71 again and the same control is repeated.

If the load becomes smaller than the tolerance (step 75: NO), current position information of the tip is obtained (step 76), the current position is set as a target value of the position to be controlled of the tip of the inserting portion (step 77), and the position of the tip of the inserting portion is controlled to keep the position (step 78).

### Third Embodiment

Although the load detecting unit is a tactile sensor which is directly provided at the tip of the inserting portion in the first and the second embodiments, the load detecting unit is not limited to the same.

In the present embodiment, the load detecting unit is a measuring unit which measures a driving current for driving the driving unit.

FIG. 7 illustrates a state of the wire when external load is applied thereto. The tip of the inserting portion should be at the position of the point A′ in a normal situation, but is pressed in the direction of the arrow C due to the contact with peripheral tissue and has been at the position of the point A.

The drive control unit 17 controls the inserting portion so that the position of the tip of the inserting portion becomes the position of A′ and, therefore, larger load than usual is applied to the tip of the inserting portion. In the case in which the wires 4A and 4B are at positions and shapes as illustrated in FIG. 7, the wire 4A has been drawn and the wire 4B has been taken out both under certain tension.

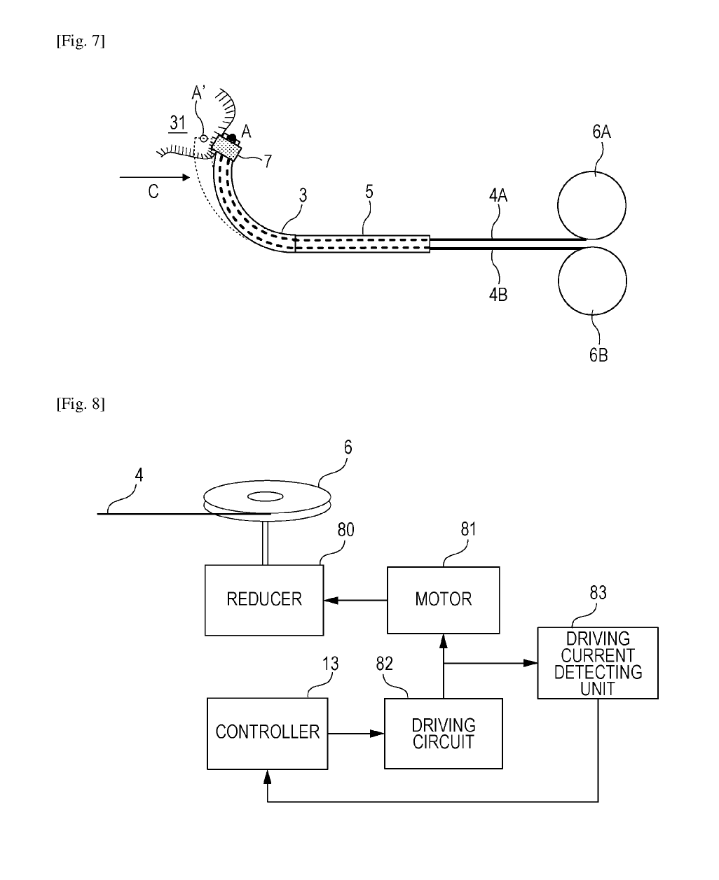

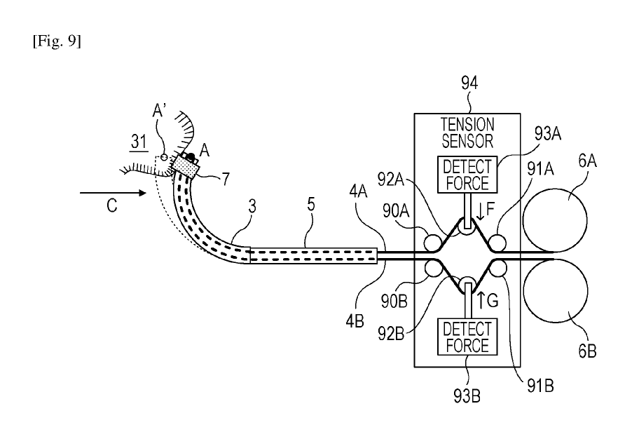

As described in FIG. 3 and FIG. 7, the wires 4A and 4B are configured to be taken up and drawn by pulleys 6A and 6B, respectively. As illustrated in FIG. 8, each of the pulleys 6 is attached to a reducer 80 and a motor 81, both of which are driving sources. The motor is connected further to a driving circuit 82 for driving. A driving current detecting unit 83 is provided in the driving circuit 82 to detect a driving current of the motor 81.

When the external force is applied in the direction of the arrow C, tension in the driving wire 4A is reduced and the driving current is reduced and, on the other hand, since the driving wire 4B is drawn and tension in the driving wire 4B is increased, the driving current is increased. The driving current detecting unit 83 detects the reduction and increase in the driving current and the controller 13 determines that the reduction and the increase respectively have exceeded predetermined threshold values to know overload has been applied at the tip of the inserting portion.

### Fourth Embodiment

The present embodiment is the same with other embodiments except that the load detecting unit is a tension meter which measures tension.

With a tension sensor 94 which has a configuration illustrated in FIG. 9, overload at the tip of the inserting portion may be detected by detecting an event in which tension applied to each of the wires 4A and 4B has exceeded predetermined thresholds.

In FIG. 9, rollers 90A, 90B, 91A, 91B, 92A and 92B are disposed on paths of the wires 4A and 4B and the force of the rollers 92A and 92B in the direction of arrows F and G are detected by force detecting units 93A and 93B. Therefore, tension applied to the wires 4A and 4B may be detected. In the case of the tension applied to the wire 4B becomes low, the force in the direction of an arrow C is reduced and the case of the tension applied to the wire 4A becomes high, the force in the direction of the arrow C is increased. On the basis of the detected tension information, the controller 13 determines that the tension applied to the wires 4A and 4B has exceeded the threshold value, respectively, and detects overload at the tip of the inserting portion.

### Fifth Embodiment

Another embodiment in which external load is applied as illustrated in FIG. 7 will be described.

In this case, in spite of having been driven under a driving condition in which the tip of the inserting portion should be positioned at the position of A′ in response to the instruction from the controller, the position represented by the inserting portion shape detecting unit 12 is A.

It is also possible to determine that the inserting portion is not able to arrive at the target position due to physical load applied at the tip thereof by obtaining tip position and posture information of the inserting portion on the basis of information detected by the inserting portion shape detecting unit 12 and detecting difference between the target instructed position and the actual position.

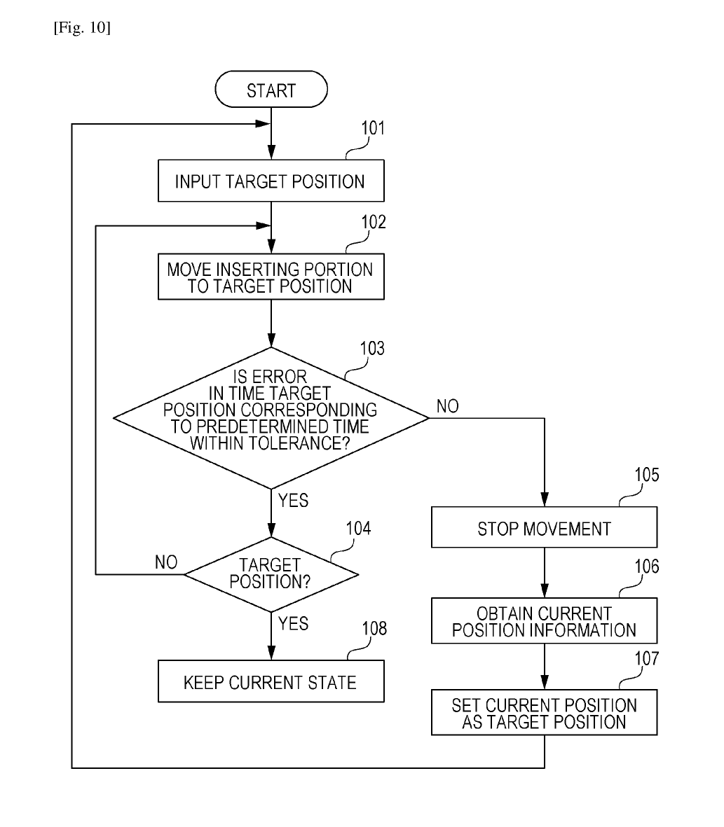

A flowchart in this case is illustrated in FIG. 10.

A target position is input from an input device (not illustrated) connected to the controller 13 (step 101) and the inserting portion 1 starts movement toward the target position (step 102).

Next, the controller 13 calculates the tip position of the inserting portion on the basis of the information from the inserting portion shape detecting unit 12 at predetermined time intervals with respect to time necessary for the movement to the target position, and compares an error between the calculated position information and the position information equivalent to the predetermined time (step 103).

Here, if the error between the calculated position information and the position information equivalent to the predetermined time is not equal to or smaller than a tolerance (step 103: NO), it is determined that the load has exceeded the tolerance in an overload determination unit (not illustrated) which is incorporated in the controller 13.

Almost at the same time, when it is determined, by the overload determination unit which is incorporated in the controller 13, that the size of the load has exceeded the tolerance, the controller 13 instructs the drive control unit 17 to stop the movement (step 105).

Here, a parameter necessary to retain the current posture is calculated in a posture retaining unit 16 on the basis of information 15 from the inserting portion shape detecting unit 12 and current position information is obtained (step 106).

The obtained current position information is set to be the target position (step 107).

An instruction is issued to the drive control unit on the basis of the parameter necessary to retain the posture. The tip of the inserting portion is controlled so that the current posture of the insert portion is kept and the insert portion is stopped at the current position.

### Sixth Embodiment

In the present embodiment, the load detecting unit is disposed at any position between the tip and the base end of the deformable portion. Except for that, the present invention is the same as any of the first to the fifth embodiments.

Since the load detecting unit is disposed at any position between the tip and the base end of the deformable portion, when load is applied from the peripheral tissue to between the tip and the base end of the deformable portion, an operator or an automatic control unit may recognize that the load has been applied and may manipulate the deformable portion so that the load from the peripheral tissue becomes small. Note that “manipulating the deformable portion so that the load from the peripheral tissue becomes small” includes manipulating the deformable portion to avoid collision with the peripheral tissue.

Desirably, the load detecting unit is disposed at the extreme value when the deformable portion is deformed. The extreme value is a portion at which the deformable portion is easily brought into contact with the peripheral tissue.

In particular, as illustrated in FIGS. 3 and 7, when the deformable portion is bent in one direction like the character of “C,” the load detecting unit is disposed at a position corresponding to the extreme value of the character of C. When the deformable portion is bent in two directions like the character of “S,” the load detecting unit(s) are disposed at one or both of positions corresponding to the two extreme values of the character of S. When the load detecting units are disposed at both of the positions corresponding to the two extreme values of the character of S, a plurality of load detecting units are disposed between the tip and the base end of the deformable portion. If the plurality of load detecting units are disposed, it is easy to specify the location to which the load is applied.

The load detecting unit(s) are disposed at the extreme value(s) in the present embodiment. However, as long as the load detecting unit(s) are disposed between the tip and the base end of the deformable portion, the load detecting unit(s) are not necessarily disposed at the extreme value(s). In addition to the portion between the tip and the base end of the deformable portion, the load detecting unit(s) may be disposed at the tip or at the tip and the base end. The load detecting units may be arranged spaced apart one another along a direction from the tip toward the base end.

If the deformable portion includes a plurality of extreme values, the deformable portions may include two kinds (three or more kinds if there are three or more portions equivalent to the extreme values) of wires of different lengths. In such a case, one end of a shorter wire is connected to a position between the tip and the base end of the deformable portion and the other end of the shorter wire is connected to the driving unit, and one end of a longer wire is connected to the tip and the other end is connected to the driving unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-124505, filed May 31, 2012 and No. 2012-169756, filed Jul. 31, 2012 which are hereby incorporated by reference herein in their entity.

### REFERENCE SIGNS LIST

1 inserting portion
2 driving unit
3 bending portion
4, 4A and 4B control wires
6, 6A, and 6B driving pulleys
7 tactile sensor
11 inserting portion load detecting unit
12 inserting portion shape detecting unit
13 controller
17 drive control unit

1. A medical instrument comprising:
a deformable portion;
a wire configured to deform the deformable portion;
a driving unit configured to drive the wire;
a drive control unit configured to control the driving unit; and
a load detecting unit configured to detect load applied to the deformable portion,
wherein when the load detected by the load detecting unit exceeds a threshold value, the drive control unit controls the driving unit to retain posture of the deformable portion.

2. The medical instrument according to claim 1, wherein the wire is inserted through the deformable portion outside the center of a section of the deformable portion.

3. The medical instrument according to claim 1, further comprising an image pickup unit and an illuminating unit at a tip of the deformable portion.

4. The medical instrument according to claim 1, wherein:
the load detecting unit is disposed at tip of the deformable portion; and
the load detecting unit is a measuring unit which is configured to measure pressure of the load detecting unit.

5. The medical instrument according to claim 4, wherein a plurality of the load detecting units are arranged spaced from one another at the tip of the deformable portion.

6. The medical instrument according to claim 1, wherein the load detecting unit is a measuring unit configured to measure a current for driving the driving unit.

7. The medical instrument according to claim 1, wherein the load detecting unit is a measuring unit configured to measure tension applied to the wire.

8. A medical instrument comprising:
a deformable portion;
a wire configured to deform the deformable portion;
a driving unit configured to drive the wire;
a drive control unit configured to control the driving unit; and
a load detecting unit configured to detect load applied to the deformable portion,
wherein the load detecting unit is disposed between the tip of the deformable portion and a base end.

9. The medical instrument according to claim 8, wherein a plurality of load detecting units are disposed between the tip of the deformable portion and the base end.

10. The medical instrument according to claim 8, wherein the load detecting unit is disposed also at the tip.

11. A medical instrument comprising:
a deformable portion;
a wire configured to deform the deformable portion;
a driving unit configured to drive the wire;
a drive control unit configured to control the driving unit; and
a load detecting unit configured to detect load applied to the deformable portion,
wherein when the load detected the load detecting unit exceeds a threshold value, the drive control unit controls the driving unit to change the deformable portion so that the load becomes small.
